(19) United States
(12) Patent Application Publication
(10) Pub. No.: US 2014/0021088 A1
(43) Pub. Date: Jan. 23, 2014

(54) PACKAGE CONTAINING A MEDICAL PRODUCT FOR TREATING CARTILAGE DAMAGE IN HUMANS OR ANIMALS

(71) Applicants: Silke Konig, Rottweil (DE); Bernd Blender, Tuttlingen (DE)

(72) Inventors: Silke Konig, Rottweil (DE); Bernd Blender, Tuttlingen (DE)

(73) Assignee: Aesculap AG, Tuttlingen/Donau (DE)

(21) Appl. No.: 13/978,022

(22) PCT Filed: Jan. 10, 2012
(86) PCT No.: PCT/EP2012/050314

§ 371 Date: Sep. 16, 2013

(30) Foreign Application Priority Data
Jan. 11, 2011 (DE) 10 2011 002 536.7

(51) Int. Cl. A61F 2/00

(52) U.S. Cl. CPC A61F 2/0095
USPC 206/570; 206/438

(57) ABSTRACT

A package in the form of a bag containing a medical product, in particular for use in the regenerative treatment of cartilage damage in humans or animals, the package having a component preferably in the form of a port, to fill the package with a liquid, preferably a cell-containing liquid.

### TECHNICAL FIELD

This disclosure relates to a package containing a medical product, wherein the medical product is provided in particular for the regenerative treatment of cartilage damage in humans or animals, preferably meniscus damage.

### BACKGROUND

The meniscus is a complicated ring-shaped or crescent-shaped cartilaginous tissue which, in contrast to an articular disk, only partly divides the joint cavity. The internal meniscus (meniscus medialis) and the external meniscus (meniscus lateralis) of the knee joint are formed as a pair of C-shaped or semi-lunar fibrocartilaginous disks. Their role is to stabilize the knee joint. They do this by compensating for lack of congruence between the articulating bones of the joint, namely the femur and the tibia, by taking up compressive forces and shear forces.

Since the internal meniscus, in contrast to the external meniscus, is fused firmly to the joint capsule and to the internal ligament, it is in principle more prone to injury than the external meniscus.

Meniscus injuries, for example, after falls and/or accidents, are generally manifested in the form of tears which can be located on the convex outer aspect of the meniscus, on the concave inner aspect of the meniscus, or therebetween.

Whereas tears on the outside can generally be sutured, torn tissue on the inner aspect of the meniscus is scraped out or cut away. A problem is that the remaining meniscus is generally no longer able to replace the scraped-out or resected tissue. In addition, partial removal of the meniscus by surgery can result in continuing degeneration of the remaining meniscus, thereby increasing the risk of the affected patients suffering from arthrosis.

For this reason, regenerative methods have increasingly been employed in recent years for the treatment of meniscus damage. The aim of these methods is to restore the original function of the meniscus by stimulating the formation of new cartilaginous tissue. In this way, arthrosis can in principle be prevented, or its clinical manifestation at least delayed.

However, for patients with very extensive meniscus damage, the only possible treatment is often to completely remove the damaged meniscus and replace it either with a donor meniscus or a so-called “regenerative implant,” i.e., an implant that induces or promotes the formation or synthesis of new tissue, in particular cartilaginous tissue. However, because of their allogenic nature, there are some reservations concerning donor menisci as regards their immunogenicity and their long-term behavior in the body of the recipient.

To induce or promote the new formation of endogenous cartilaginous tissue, implant structures provided for regenerative treatment are generally inoculated with autologous cartilage cells (chondrocytes) and then implanted back into the patient as regenerative implants. A basic difficulty in this case is that there is generally only a limited amount of autologous chondrocytes available for the inoculation of the implant structures. In addition, the implant structures provided for regenerative cartilage treatment are very often characterized by a heterogeneous composite structure. Overall, therefore, even with prolonged incubation or cultivation with cartilage cells, there is some risk of the implant structures being only incompletely colonized which can have an adverse effect on the new formation of endogenous cartilaginous tissue.

There is, therefore, a need to create a package in which a medical product is made available that is suitable in particular for the regenerative treatment of tissue damage, in particular cartilage damage, for example, meniscus damage and that avoids known disadvantages. The package should ensure that a medical product contained within the package is colonized with cells and/or active substances very simply and efficiently and, in particular, in a homogeneous manner.

### SUMMARY

We provide a package including a bag and containing a medical product for the regenerative treatment of cartilage damage in humans or animals, and a port to fill the package with a cell-containing liquid.

We also provide a medical kit including a package with a component to fill the package with a cell-containing liquid and a medical product, wherein the medical product is contained in the package.

### BRIEF DESCRIPTION OF THE DRAWINGS

FIG. 1 shows an example of one of our packages.

FIG. 2 shows another example of one of our packages.

FIG. 3 shows an enlarged view of the medical product contained in the package of FIG. 2.

FIG. 4 shows a side view of the product shown in FIGS. 2 and 3.

### DETAILED DESCRIPTION

We provide a package, in particular in the form of a bag, preferably in the form of an infusion bag, containing a medical product, in particular for the regenerative treatment of tissue damage in humans or animals, preferably damage to connective tissue, more preferably damage to supporting tissue, particularly preferably damage to cartilaginous tissue, especially preferably damage to the meniscus. The package also has a component to fill the package with a liquid.

The package, in particular the component provided to fill the package, hereinafter also referred to as the “filling component,” particularly advantageously ensures that a medical product still in the packaged state is incubated, in particular wetted and/or saturated, with a liquid. In other words, the package can be used particularly advantageously as a kind of incubation vessel for the product and for a liquid with which the product is to be incubated.

The liquid is preferably a liquid with a medical action, in particular a therapeutic action. In this way, it is possible for the medical product to be provided with certain medical or therapeutic properties specific to the indication before it is removed from the package.

The liquid may be a liquid that contains active substance and/or cells, preferably a liquid that contains cells. For example, if the liquid contains cells such as autologous somatic cells, then the package acts particularly advantageously as a kind of culture vessel for the medical product contained in the package and (after the package has been filled with the cell-containing liquid) for the cells contained in the package. To cultivate cells in the package, provision can be made, for example, to fill the package additionally with suitable nutrient solutions. Suitable active substances and/or cells will be discussed in more detail below.

Preferably, the package is designed as a bag. The package is particularly preferably a plastic bag.

Preferably, the package is produced from a film, in particular a flat film or tubular film. The film can be designed as a single-ply or multi-ply film, in particular a two-ply or three-ply film.

Particularly preferably, the package is produced from a plastic film, in particular a two-ply plastic film.

The package may be a plastic film bag, in particular a two-ply plastic film bag.

The package, or the plastic film mentioned above may be produced from a plastic chosen from the group consisting of polyolefins, polyamides, copolymers thereof, and mixtures, in particular blends, thereof.

The package or the plastic film is particularly preferably produced from a plastic chosen from the group consisting of polyethylene, high-density polyethylene (HDPE), polypropylene, high-density polypropylene (HDPP), copolymers thereof, in particular ethylene vinyl alcohol, and mixtures, in particular blends, thereof.

The package is expediently a closed, in particular sealed, preferably welded package. It is particularly advantageous if the package is closed to be germ-proof, gas-tight and, in particular, moisture-proof.

Particularly preferably, the package is welded. The package is welded preferably by methods of welding of plastics, for example, heated-tool welding, induction welding, laser welding, IR welding, hot-gas welding, extrusion welding, high-frequency welding, ultrasonic welding, vibration welding or the like. Such welding methods are known per se and, therefore, these methods are not discussed in detail.

Provision is made in particular that the package is welded along a fold, in particular along a fold running at the edge.

The filling component may have a plastic body. The plastic body is preferably welded in between at least two, preferably two, plies, in particular between at least two, preferably two, plastic film plies, of the package.

The component provided to fill the package may be a port. In particular, the port can have a plastic body preferably welded in between two plies, in particular between two plastic film plies, of the package, preferably thus forming a port weld area.

The plastic body is typically produced from a polyolefin, for example, polypropylene.

Particularly preferably, the package has a port weld area and a fold weld area, in particular a fold weld area running at the edge. In the area of a port lying between at least two, in particular two, plies of the package, the fold weld area preferably merges, generally seamlessly, into a port weld area.

Alternatively, the package may be designed as a blister pack, in particular as a thermoformed blister pack.

The blister pack generally has a shaped plastic part, in particular a thermoformed plastic part, preferably with a housing shape.

The shaped plastic part can be formed from a plastic material such as polyethylene terephthalate (PET) and/or polycarbonate.

Moreover, the shaped plastic part can in particular be film-like or configured as a film.

The blister pack may have a gas-permeable and, in particular, germ-proof cover layer. The cover layer acts particularly advantageously as a kind of sterilization window in which it permits passage of sterilization gases, for example, ethylene oxide gas, into the interior of the package. After the sterilization procedure has been completed, the sterilization gas can be removed again from the package, for example, by applying a vacuum or negative pressure by virtue of the gas permeability of the cover layer.

Particularly preferably, the package has a gas-permeable and, in particular, germ-proof cover layer with a web-like structure, in particular a structure like a paper web.

The cover layer can in particular be formed from a polyolefin, for example, polyethylene, in particular high-density polyethylene (HDPE), polypropylene, in particular high-density polypropylene (HDPP), copolymers thereof, and mixtures, in particular blends, thereof.

It may be preferable if the cover layer is film-like or configured as a film (cover film).

Particularly preferably, the cover layer is a web-like, in particular paper-web-like, fibrous functional textile, preferably of thermally welded fibers. A suitable cover layer can be produced, for example, from a material commercially available under the name Tyvek®. This is a paper-web-like fibrous functional textile made from thermally welded fibers of high-density polyethylene (HDPE).

The blister pack may have a gas-tight and, in particular, moisture-proof top layer. This permits dry storage of the medical product in the blister pack.

The top layer may be composed of aluminum or of a composite material with aluminum. A top layer composed of or with aluminum alloys is likewise possible.

The top layer can be a composite material with a layered structure. Preferably, the structure has a sequence of layers chosen from the group consisting of polyethylene/aluminum/polyethylene terephthalate, polyvinyl chloride/aluminum/polyamide, polypropylene/aluminum/paints, polyethylene terephthalate/aluminum/polypropylene, and paints/aluminum/polyethylene terephthalate.

It may be preferable if the top layer is film-like or is configured as a film (top film).

Particularly preferably, the blister pack has a gas-permeable and germ-proof cover layer and a gas-tight and, in particular, moisture-proof top layer, with the cover layer preferably being covered or sealed by the top layer.

The package preferably has a wall which surrounds the filling component, in particular a plastic body of the filling component, with a form fit and, in particular, sealingly.

Advantageously, the component for filling the package may be designed such that it can be clamped.

The component to fill the package is preferably produced from a material, for example, rubber that can be pierced by an injection needle. The filling component can therefore be a rubber stopper, for example. Alternatively, the filling component can also be designed as a septum.

The component to fill the package may be closed by a material that can be pierced with an injection needle, preferably by a membrane or a septum.

The medical product may be stored under vacuum or at a negative pressure in the package. This has the advantage that, when the package is filled with a liquid, some of the liquid is immediately drawn into the interior of the package by virtue of the vacuum or negative pressure prevailing in the package, until a pressure compensation is established. It is thus possible to achieve complete saturation, in particular an efficient and preferably homogeneous cellular colonization, of the medical product. This is a decisive advantage, especially in view of the limited availability of autologous somatic cells to charge the product.

For example, if the package is filled with the liquid content of a syringe, some of the syringe content is drawn automatically out of the syringe into the interior of the package after the syringe needle has pierced the filling component which, in this instance, is expediently formed from a material that can be pierced by an injection needle, or is closed by such a material.

The medical product may be present a compressed state on account of a vacuum or negative pressure prevailing in the package. As the package is being filled with liquid, a relaxation or expansion of the medical product can particularly advantageously also increase the suction effect of the product and, for example, accelerate the complete and, particular, homogeneous addition of cells and/or active substances to the product.

Furthermore, it is preferable if the medical product in the package is sterilized, preferably sterilized by γ radiation. As a result, an incubation of the product with a liquid, in particular with a liquid that contains cells and/or active substance, is possible under sterile or aseptic conditions.

Further advantageously, the package may contain, in addition to the medical product, a liquid, in particular in the form of a solution, dispersion or suspension. As has already been mentioned, the liquid is preferably a liquid with a medical action, in particular a therapeutic action.

The liquid preferably contains active substances and/or cells. Particularly preferably the liquid is a cell-containing liquid, in particular a cell-containing suspension.

The active substances can be chosen from the group consisting of antimicrobial substances, in particular antibiotic substances, disinfecting agents, anti-inflammatory substances, analgesic agents, odor-inhibiting substances, anti-proliferative substances or substances inhibiting cell division, substances promoting cell growth, cell-recruiting agents, cell-differentiating agents, substances promoting tissue growth, chondro-inductive substances, i.e., substances stimulating the growth of cartilaginous tissue or the regeneration of cartilaginous tissue, and mixtures thereof.

The cells are preferably autologous cells, in particular autologous somatic cells. Autologous cells have the advantage that severe immunological reactions, in particular graft rejection, can be avoided.

However, the cells can also be of allogenic origin.

The liquid preferably contains cells chosen from the group consisting of chondrocytes, chondroblasts, osteoblasts, synovial cells, fibroblasts, cells of the periosteum, cells of the perichondrium, stromal cells, in particular mesenchymal stromal cells, precursor cells thereof stem cells thereof stem cells, in particuhr mesenchymal stem cells, and combinations thereof.

Furthermore, the medical product can be designed to be compressible, in particular reversibly compressible. A “reversibly compressible” product is to be understood as a product which can be converted to a compressed state under a load, in particular a pressure or negative pressure or vacuum, and which, after removal of the load, adopts or substantially adopts its original shape and size again.

The product is expediently biocompatible, in particular with respect to cells, and is preferably absorbable, in particular bioabsorbable, i.e., absorbable in vivo.

The medical product is preferably designed as an absorbent body or preferably has an absorbent body. The absorbent body can be sponge-like or foam-like.

The medical product may have a foam-like component, membrane-like component and/or fibrous component. Preferably, the medical product has a foam-like component and a membrane-like component and/or a fibrous component.

foam-like component is preferably formed from a polymer, in particular a synthetic polymer, in particular a plastic. A polymer mixture is likewise possible.

The foam-like component is preferably a polymer foam, in particular a synthetic polymer foam, preferably a plastic foam.

Preferably, the foam-like component is a foamed polymer or, if appropriate, a foamed polymer mixture. A “fbamed polymer” or a “foamed polymer mixture” is to be understood as a polymer or a polymer mixture which can be produced by foaming methods known per se, for example, the reaction foam molding method or in the manner of a reaction foam molding method, as a foam or foam-like matrix or structure.

Particularly preferably, the polymer is polyurethane. The polymer can be in particular a linear or branched polyurethane. Additionally or alternatively, the polymer can be an aliphatic or aromatic polyurethane. A linear and, in particular, aliphatic polyurethane is preferred.

The polymer may be a polyurethane which, proceeding from at least one polyol component and at least one polyisocyanate component, is produced or can be produced preferably by a foaming method, in particular by a reaction foam molding method or in the manner of a reaction foam molding method.

A “polyol” component is to be understood as an alcohol compound having two hydroxy groups (diol component) or more hydroxy groups. Correspondingly, a “polyisocyanate” component is to be understood as an isocyanate compound having two isocyanate groups (diisocyanate component) or more isocyanate groups.

Particularly preferably, the polymer is a polyurethane which, proceeding from a polyol component chosen from the group consisting of ethylene glycol, diethylene glycol, propylene trimethylolpropane, glycerol, neopentyl glycol, 1,3-propanediol, 1,4-butanediol, 1,6-hexanediol, polycaprolactone diol, polycaprolactone triol, polyethylene glycol and mixtures thereof, and a polyisocyanate component chosen from the group consisting of diphenylmethane diisocyanate, toluene-2,4-diisocyanate, naphthylene diisocyanate, hexamethylene diisocyanate, isophorone diisocyanate, 4,4′-diisocyanatodicyclohexyl methane, butane diisocyanate, lysine diisocyanate and mixtures thereof, is produced or can be produced by nucleophilic polyaddition.

The foam-like component expediently has a porous structure, in particular an open-pore structure. The foam-like component preferably has a pore size of 10 to 450 μm, in particular 30 to 350 μm, preferably 50 to 250 μm. These pore sizes have proven to be particularly advantageous as regards the ingrowth or inward proliferation of cells, tissue and supply vessels.

The foam-like component may have areas with different pore structures, in particular different pore sizes and/or geometries. In particular, the foam-like component can have a pore gradient.

The foam-like component may have a femoral surface, a tibial surface, and a join capsule surface. A “femoral” surface is to be understood as a surface of the foam-like component which, after the implantation of the product into a knee joint, faces the femur or the femoral condyles. A “tibial” surface is intended to signify a surface of the foam-like component which, after the implantation of the product into a knee joint, faces the tibia or the tibial condyles. A “joint-capsule” surface is to be understood as a surface of the foam-like component which, after the implantation of the product, faces the joint capsule (integument) of the knee joint.

Provision can also be made that the foam-like component has pore sizes decreasing in the direction of the femoral surface and/or in the direction of the tibial surface. In particular, the femoral surface and/or the tibial surface can have such small pore sizes that inward cellular proliferation is no longer possible. In general, these are pore sizes of <1 μm. Small pore sizes of this kind make the femoral surface and/or the tibial surface more slidable, which results in a smoother articulation with the femur and/or the tibia. In this way, it is also possible to prevent cell loss via the femoral and/or tibial surface.

Only the joint-capsule surface of the foam-like component need be formed with open pores in one example. This permits a channeled inward proliferation of cells from the joint capsule of a knee joint into the foam-like component, capsule is generally a cell-rich region that borders the outer area of a meniscus.

The foam-like component may have a proportion of 70 to 98% by weight, in particular of 80 to 97% by weight, preferably 85 to 95% by weight, relative to the total weight of the product.

Particularly advantageously, the foam-like component has channels or channel-like structures, preferably with a porous inner wall. The channels or channel-like structures can have an internal diameter (clear width) of 100 to 600 μm, in particular of 200 to 500 μm, preferably about 500 μm. The channels or channel-like structures particularly advantageously provide guide rails for inwardly proliferating cells, inwardly proliferating tissue and/or inwardly proliferating supply vessels, in particular blood vessels.

The membrane-like component preferably has a tear-resistant structure with tensile strength and, in particular, allows the medical product to be anchored safely in the body of a patient, in a manner secure against displacement and dislocation. The membrane-like component is able, in particular, to at least partially absorb forces which act on the foam-like component, and which can typically occur in the body of a patient, for example, shear forces, thereby preventing mechanical damage to the foam-like component. In addition, the membrane-like component preferably has a guide rail function in respect of cells, for example, chondrocytes, connective tissue, for example, peripheral connective tissue, and/or natural supply vessels, for example, blood vessels.

The membrane-like component can in particular be non-porous or substantially non-porous. It is also possible for the membrane-like component to have such small pore sizes, for example, <1 μm that cellular penetration or infiltration of the membrane-like component is not possible, and the membrane-like component therefore acts as a barrier impenetrable to cells.

The membrane-like component may have a proportion of 1.5 to 20% by weight, in particular 2 to 15% by weight, preferably 2 to 10% by weight, relative to the total weight of the product.

Particularly preferably, the membrane-like component is contained partially, particular only partially, in the foam-like component and/or lies partially, in particular only partially, embedded in the foam-like component. In particular, the membrane-like component can be integrated partially, particular only partially, in the structure of the foam-like component.

Particularly preferably, the membrane-like component protrudes into the foam-like component or the structure thereof. In other words, it is particularly preferable if the membrane-like component protrudes from the foam-like component or the structure thereof, or if the membrane-like component is partially not surrounded or enclosed by the foam-like component.

it is also preferable if the membrane-like component extends, preferably continuous-ly, in the longitudinal direction of the foam-like component.

The membrane-like component preferably protrudes, preferably in the longitudinal direction of the foam-like component, along a joint-capsule surface of the foam-like component.

The membrane-like component may have apertures, in particular in the form of openings, holes, perforations or the like. Along the apertures, the membrane-like component is preferably penetrated by the foam-like component. Thus possible to achieve a firm integration of the membrane-like component in the foam-like component.

membrane-like component may be of biological origin, in particular of xenogenic origin, preferably of animal origin. For example, the membrane-like component can be of porcine, bovine and/or equine origin. The membrane-like component is preferably of bovine origin.

The membrane-like component may have a fibrous structure or a fiber structure.

The membrane-like component preferably has a protein, in particular a fibrillar or fibrous protein, preferably an extracellular protein.

The protein is preferably contained as main constituent in the membrane-like component. Thus, the membrane-like component can have a protein content of more than 50% by weight, in particular of more than 75% by weight, preferably of more than 90% by weight, relative to the total weight of the membrane-like component.

The membrane-like component is particularly preferably formed from the protein.

The protein can be chosen in particular from the group consisting of collagen, elastin, reticulin, fibronectin, gelatin, salts thereof, derivatives thereof, and mixtures thereof.

Preferably, the protein is collagen, in particular fibrous or fibrillar collagen, preferably chosen from the group consisting of type I collagen, type II collagen, type III collagen, type V collagen, type XI collagen, salts thereof, derivatives thereof, and mixtures thereof. Type I collagen is particularly preferred.

The membrane-like component is preferably a membrane, in particular a synthetic or biological membrane. The membrane-like component is particularly preferably a protein membrane, in particular collagen membrane. A particularly preferred membrane-like component is the collagen membrane sold commercially by the applicant under the name Lyoplant®. Regarding further features and advantages of the protein, in particular collagen, reference is made in full to the preceding comments.

The membrane-like component may be a biological tissue, in particular a layer of tissue, for example, the submucosa. A “biological” tissue is to be understood as a tissue which is found in particular in animal organisms or can be produced by tissue engineering.

The membrane-like component may be an extracellular matrix (ECM), in particular chosen from the group consisting of pericardium, peritoneum, small intestine submucosa, stomach submucosa, bladder submucosa, uterus submucosa, serosa, and combinations thereof.

Particularly preferably, the membrane-like component is chosen from the group consisting of pericardium, fibrous pericardium, serous pericardium, epicardium, squamous epithelium, serosa, muscle, for example, myocardium, and combinations thereof.

Particularly preferably, the membrane-like component is pericardium, in particular animal pericardium. The pericardium can, for example, be of porcine, bovine and/or equine origin. Preferably, the membrane-like component is bovine pericardium.

The fibrous component preferably likewise acts as a guide rail for cells growing or proliferating inward into the foam-like component.

The fibrous component is preferably contained in the foam-like component, in particular exclusively in the foam-like component.

The fibrous component preferably has the same material as the membrane-like component or is formed from the same material as the membrane-like component.

Advantageously, the fibrous component involves hydrophobic fibers. Hydrophobic fibers have the advantage that cells can better adhere to them, as a result of which the guide rail function with respect to cells can be improved.

The fibrous component may involve fibers containing biopolymers, in particular fibers containing polysaccharides, for example, fibers containing mucopolysaccharides, and/or fibers containing protein, preferably fibers containing collagen.

The fibrous component preferably involves fibers with a protein content, preferably a collagen content, in particular a collagen type I content, of more than 50% by weight, in particular of more than 60% by weight, preferably of more than 70% by weight, particularly preferably of about 75% by weight, relative to the individual weight of the fibers.

The fibrous component may involve fibers of biological origin, in particular of xenogenic origin, preferably of animal origin. For example, the fibrous component can involve fibers of porcine, bovine and/or equine origin. Bovine fibers are particularly preferred.

Preferably, the fibrous component is made of Lyoplant® fibers.

Preferably, the fibrous component involves pericardial fibers, epicardial fibers, myocardial fibers, and combinations thereof.

The fibrous component preferably has a proportion of 1 to 10% by weight, in particular 1.5 to 7.5% by weight, preferably 2 to 6% by weight, relative to the total weight of the product.

The product may consist of a foam-like component, a membrane-like component and a fibrous component. For further features and advantages, reference is made in full to the foregoing description.

Advantageously, the product may be charged with cells, in particular autologous cells, preferably autologous somatic cells. As regards suitable cells, reference is made in full to the foregoing description.

Furthermore, the product can be charged with active substances. As regards suitable active substances, reference is made in full to the foregoing description.

product, in particular the foam-like component, can be present in different shapes and sizes. For example, the medical product can be disk-shaped, in particular as a circular disk, cylindrical, in particular as a flat cylinder, wedge-shaped or strip-shaped.

In particular, the product can have a curved or curvilinear profile.

medical product, in particular the foam-like component, is preferably semi-circular, C-shaped, crescent-shaped or semi-lunar. In particular, the product can be formed as a semi-circular, C-shaped, crescent-shaped or semi-lunar wedge or as a semi-circular, C-shaped, crescent-shaped or semi-lunar disk.

The medical product can in principle be a product chosen from the group consisting of surgical implants, hemostatics, wound dressings, medical meshes, vein sheaths, vascular prostheses, in particular arterial prostheses, stents, and stent-grafts. Surgical meshes, for example, hernia meshes, prolapse meshes and/or incontinence bands.

In particular, the medical product can be a combined or composite implant.

Preferably, the medical product is formed as an implant, in particular for use in the regeneration and/or reconstruction of biological tissue (regenerative implant), in particular connective tissue, preferably support tissue, particularly preferably cartilaginous tissue, in particular meniscal tissue.

Particularly preferably, the medical product is an implant for cartilage replacement or partial cartilage replacement, in particular an implant for meniscus replacement or partial meniscus replacement, preferably for the internal and/or external meniscus of a knee joint.

Finally, we provide a medical combination, preferably a medical kit, comprising a package with a component for filling the package (filling component) with a liquid, preferably a cell-containing liquid, and a medical product. The package and the product can in principle be separate from each other. In particular, the product can be transferred into the package only when, for example, autologous somatic cells have been removed from a patient with tissue damage, for example, cartilage damage. However, it is preferable if the medical product is contained in the package, i.e., is part of the package. To avoid unnecessary repetition, for further features and advantages of the package and/or of the medical product, reference is made in full to the foregoing description.

Further features and advantages of our packages will become clear from the following description in combination with the figures, the figure descriptions and the examples. Individual features can be implemented singly or in combination with each other. The described structures are to be understood as being merely descriptive and not as a disclosure limiting this disclosure in any way. All of the figures are hereby incorporated by express reference into the content of this description.

### 1. Preparation of a Product for Replacement or Partial Replacement of a Meniscus

### 1.1 Preparation of Bovine Pericardium Membrane

The membrane was perforated by a needle (21 G×2). The membrane was then dried in vacuo (2-10 mbar) for at least six hours. Thereafter, the membrane was placed in a mixture of polycaprolactone diol (Mₙ=2000) and isophorone diisocyanate in a molar ratio of 9:10 at 50° C. After 90 minutes, the membrane and the mixture were transferred into a polyethylene weighing boat, in which the membrane was fixed by a PTFE-encased stirring bar. The membrane was left there for a total of two hours, after which the membrane was placed in a two-part silicone mold (molding tool).

### 1.2 Placing the Pericardium Membrane in a Molding Tool

A two-part silicone mold with a semi-circular cavity was maintained at 50° C. for at least one hour before introduction of the pericardium membrane. The membrane was then placed in the silicone mold and fixed with the aid of three needles (21 G×2). Thereafter, the silicone mold was fixed from the outside with an adhesive tape.

### 1.3 Preparing a Polyol Mixture

The polyols polycaprolactone diol, polycaprolactone triol, polyethylene glycol and butane diol were heated for liquefaction to a temperature of about 40° C. to liquefy them. Thereafter, the polyols were weighed into a suitable vessel, also heated to 40° C. and mixed. Subse-quently, the additives DABCO, DC3042 and lecithin were added. Thereafter, a homogeneous mixture was produced. Finally, collagen-containing fibers from the muscle layer of the heart (myocardium) were added to the mixture. The obtained mixture was once again homogenized.

### 1,4 Adding the Other Components

methylal was added as propellant. After homogenization of the mixture, diazabicycloundecene was added.

When the mixture began to was injected into the silicone mold by a 50-ml syringe. For this purpose, the silicone mold had suitable injection openings.

The silicone mold was stored for at least five hours at about 50° C. in an incubator. The closed mold was then allowed to cool for at least two hours.

The silicone mold was then opened, and the finished product carefully removed. Excess polyurethane (sprues, material in the venting channels, porous film in the interior of the semi-circular segment of the product) was removed. The product was tumbled in completely demineralized water for about two minutes and then dried under vacuum to constant weight.

The product obtained had a shape as shown schematically in FIGS. 2 to 4.

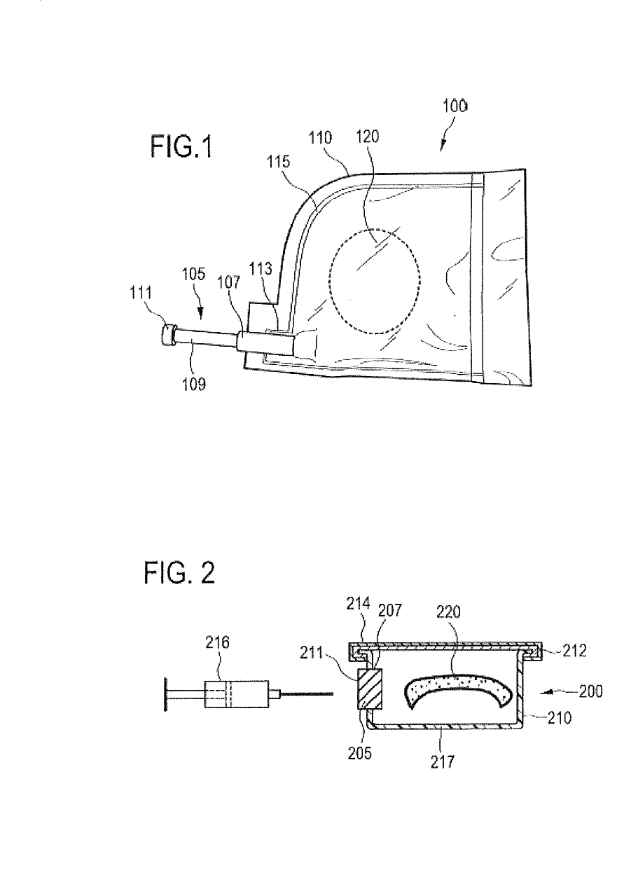

FIG. 1 shows schematically a package 100 designed as a plastic bag and containing a medical product 120.

The plastic bag 100 is produced from a plastic film 110, in particular from two plies thereof. The plastic film 110 can be a polypropylene film, for example.

To fill with a liquid, preferably with a liquid containing autologous chondrocytes, the package 100 has a filling component 105 designed as a port. The port 105 has a plastic body 107, which is welded at least partially along a port weld area 113 in the package 100. The port 105 is designed such that it can be clamped shut and, for this purpose, has a section 109 which protrudes from the plastic body 107 and which can be clamped shut or pressed together. The port 105 is covered, for example, by a septum 111. With a liquid, the needle of a syringe, for example, can be pushed into the septum 111, and the liquid can be discharged through the septum 111 into the package 100 by actuation a syringe plunger.

The port weld area 113 merges preferably seamlessly into a fold weld area 115 running at the edge (welding along a fold running at the edge).

climatically in FIG. 1 has a cylindrical shape, in particular a fiat cylindrical shape. Depending on the indication, the medical product 120 can of course also have other shapes and, in particular, sizes.

The medical product 120 is preferably a foam, in particular a polymer foam. The medical product 120 is particularly preferably a polyurethane foam or foamed polyurethane.

The medical product 120 is preferably stored in a sterile state in the package 100. It is thus possible, for example, to carry out sterile cellular colonization of the product 120 in the package 100.

In the package 100, there is preferably a vacuum or negative pressure which keeps the product 120 in a compressed state. The product 120 is particularly advantageously designed to be reversibly compressible, as a result of which its suction effect is preferably increased during the filling of the package 100 with a liquid. This allows the product 120 to be saturated as completely as possible with the liquid, and this, for example, in the case of a cell-containing liquid, can promote: very rapid and in particular homogeneous cellular loading of the product 120.

FIG. 2 shows schematically a further example of a package 200. The package 200 is designed as a thermoformed blister pack. The thermoformed blister pack 200 comprises a thermoformed plastic part 210, for example, of polyethylene terephthalate with a housing shape in which a medical product 220 is stored. The thermoformed plastic part 210 is preferably closed by a gas-permeable and in particular germ-proof cover layer 212. The cover layer 212 can be formed, for example, from the material sold commercially under the name Tyvek®. The cover layer 212 is in turn preferably covered or sealed by a gas-impermeable and in particular liquid-impermeable top layer 214, preferably made of aluminum or a composite material with aluminum. Both the cover layer 212 and also the top layer 214 are preferably formed like a film.

To fill it with a liquid, for example, with the aid of a syringe 216, the package 200 has, for example, a filling component 205 which is sealed with a septum 211 and has a plastic body 207 integrated preferably with a form fit, and in particular sealingly, in the wall 217 of the thermoformed plastic part 210.

The medical product 220 shown in FIG. 2 preferably has the shape of a semi-circular or C-shaped wedge and, in this form, is particularly suitable for the treatment of meniscus damage, in particular of a knee joint. Depending on the medical indication to be treated, other shapes are of course also conceivable for the medical product.

product 220 stored in the package 200 is also preferably sterilized and, by virtue of a negative pressure or vacuum in the package 200, can likewise be present in a compressed state.

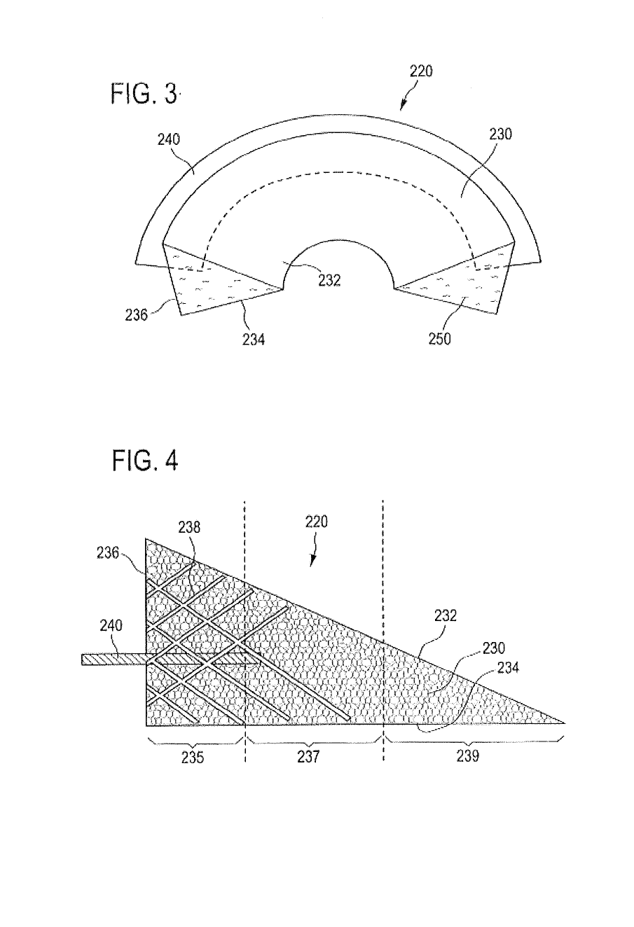

FIG. 3 shows schematically the medical product 220 from FIG. 2 in an enlarged view. The medical product 220 has a foam-like component 230, a membrane-like component 240, and a fibrous component 250.

The foam-like component 230 generally has an open-pore structure and serves preferably as a support matrix for ingrowing and proliferating cells, for example, chondrocytes, precursors thereof and/or stem cells.

foam-like component 230 is preferably a foamed polymer or a polymer foam, in particular foamed polyurethane e or a polyurethane foam.

The foam-like component 230 preferably has the shape of a semi-circular or C-shaped wedge or a semi-circular or C-shaped disk. However, depending on the particular medical indication, other shapes are also conceivable.

The product 220 shown schematically in FIG. 3 (and FIG. 2) is suitable in particular as a meniscus replacement implant or partial replacement implant, preferably as a replacement implant or partial replacement implant for the internal meniscus and/or external at meniscus of a knee joint, preferably a human knee joint.

implantation of the product 220 into a knee joint, the surface 232 preferably faces the femoral condyles and forms the femoral surface. After implantation of the product 220 into a knee joint, the surface 234 preferably faces the tibial condyles and forms the tibial surface. After implantation of the product 220, the surface 236 preferably faces the joint capsule of a knee joint and forms the joint-capsule surface.

The membrane-like component 240 protrudes, along the surface 236, from the foam-like component 230.

The membrane-like component 240 preferably has substantially the same curved profile as the foam-like component 230.

Preferably, the membrane-like component 240 is an animal membrane, for example, bovine pericardium.

The fibrous component 250 is preferably in the form of protein-containing fibers, in particular collagen-containing fibers, for example, myocardial fibers. The fibrous component 250 is preferably contained exclusively in the foam-like component 230.

As has already been mentioned in the foregoing description, both the membrane-like component 240 and also the fibrous component 250 can act particularly advantageously as a guide rail for cells, body tissues and/or natural supply vessels in the direction of the foam-like component 230. This permits a rapid, quantitative and, in particular, homogeneous cellular colonization of the foam-like component 230, a connective-tissue-based anchoring of the product 220 in a body defect zone, and/or an adequate supply of nutrients. This favors, in particular accelerates, the new formation of target tissue, preferably cartilage tissue, in a body defect zone that is to be supplied, preferably a cartilage zone.

To additionally improve cell proliferation, tissue proliferation and/or vessel proliferation into the foam-like component 230, it is possible for the foam-like component 230 to have channels or channel-like structures 238 (FIG. 4; fibrous component is not shown in FIG. 4). For example, the channels 238 can be formed only in an outer area 235 and a middle area 237 of the foam-like component 230, while a central area 239 of the foam-like component 230 is preferably free of such channels 238. Thus, the central area 239 has a more compact structure in relation to the peripheral area 235 and middle area 237, which is advantageous in particular in the case of a product 220 which is designed as a meniscus replacement or partial replacement implant and in which the central area 239 is exposed to particularly high loads, in particular shear forces.

The package particularly advantageously permits the provision of a medical product to which cells and/or active substances have been added. Preferably, the package is used to make available a product which is charged with cells, preferably autologous cells of the body, for example, chondrocytes, particular for the regeneration and/or reconstruction of tissue damage, in particular cartilage damage, for example, meniscus damage. For this purpose, the medical product stored in the package is incubated with a cell-containing liquid, generally a cell-containing suspension, via the filling component. In this way, it is possible to achieve a sufficient and, in particular, homogeneous cellular colonization of the product even during its preferably sterile storage in the package. To accelerate the cellular colonization, provision can be made, for example, to additionally fill the package with suitable nutrient solutions via its filling component.

In conclusion, the package therefore represents a filling device for the medical product contained therein, which device can be produced easily and, in particular, in a sterile state and particularly advantageously provides the physician with an advantageous and convenient possibility whereby the medical product, even during its storage in the package, can be colonized efficiently, and under germ-free conditions, with a generally only limited available amount of autologous cells, for example, chondrocytes. After the cells have multiplied sufficiently in the package, the product can be removed from the package and implanted directly into the body of a patient without the need for additional measures that could possibly adversely affect the integrity of the product, the amount of cells in the product and/or the sterility of the product.

1. A package comprising a bag and containing a medical product for the regenerative treatment of cartilage damage in humans or animals, and a port to fill the package with a cell-containing liquid.

2. The package as claimed in claim 1, which comprises a plastic bag.

3. The package as claimed in claim 1, produced from a two-ply plastic film.

4. The package as claimed in claim 3, wherein the plastic film is produced from a plastic that is selected from the group consisting of polyethylene, polypropylene, copolymers thereof, and mixtures thereof.

5. The package as claimed in claim 1, wherein the port comprises a plastic body welded in between two plastic film plies of the package.

6. The package as claimed in claim 1, wherein the package is welded along a fold running at an edge thereof.

7. The package as claimed in claim 1, wherein the package has a port weld area and a fold weld area, and the port weld area and the fold weld area merge seamlessly into each other.

8. The package as claimed in claim 1, which is a thermoformed blister pack.

9. The package as claimed in claim 8, wherein the blister pack has a gas-permeable, germ-proof cover layer with a web-like structure.

10. The package as claimed in claim 8, wherein the blister pack has a gas-tight and liquid-tight top layer of aluminum or with an aluminum-containing composite structure.

11. The package as claimed in claim 8, wherein the blister pack has a gas-permeable and germ-proof cover layer and a gas-tight and liquid-tight top layer, wherein a cover layer is covered and sealed by a top layer.

12. The package as claimed in claim 1, wherein the package has a wall which sealingly surrounds a plastic body of the port, with a form fit.

13. The package as claimed in claim 1, wherein the port is designed to be clamped.

14. The package as claimed in claim 1, wherein the port is closed by a material that can be pierced with an injection needle.

15. The package as claimed in claim 1, wherein the medical product is stored under vacuum or at a negative pressure in the package.

16. The package as claimed in claim 15, wherein the medical product is present in a compressed state on account of the vacuum or negative pressure.

17. The package as claimed in claim 1, wherein the medical product is sterilized by γ radiation.

18. The package as claimed in claim, wherein the package has, in addition to the medical product, a liquid that contains active substance and cells, or the medical product present in the package is wetted and/or saturated by a liquid that contains active substance and/or cells.

19. The package as claimed in claim 1, wherein the medical product is an implant for replacement of cartilage or partial replacement of cartilage, preferably an implant for replacement of the meniscus or partial replacement of the meniscus.

20. A medical kit comprising a package with a component to fill the package with a cell-containing liquid and a medical product, wherein the medical product is contained in the packag.

21. (canceled)
